(19) United States
(12) Patent Application Publication
(10) Pub. No.: US 2024/0369548 A1
(43) Pub. Date: Nov. 7, 2024

(54) ELECTROCHEMICAL SHOTGUN TAGGING ASSAY

(71) Applicant: Oxford University Innovation Limited, Oxford (GB)

(72) Inventors: Jason DAVIS, Oxford (Oxfordshire) (GB); Mohamed SHARAFELDIN, Dakhliya (EG); Felix FLESCHHUT, Kaufbeuren (DE)

(21) Appl. No.: 18/687,538

(22) PCT Filed: Sep. 8, 2022
(86) PCT No.: PCT/GB2022/052282

§ 371 Date: Feb. 28, 2024

(30) Foreign Application Priority Data
Sep. 10, 2021 (GB) 2112927.5

(51) Int. Cl. G01N 33/543; G01N 27/327; G01N 33/68

(52) U.S. Cl. CPC G01N 33/5438; G01N 27/3277; G01N 33/68

(57) ABSTRACT

The present invention relates to the electrochemical detection of a target protein in a sample typically comprising a protein ensemble, which utilises shotgun tagging of protein in the sample followed by selective binding of tagged, target protein and electrochemical sensing thereof.

The present invention relates to the electrochemical sensing of proteins using shotgun redox tagging.

### BACKGROUND

Assaying proteins in biological samples is essential for many applications including clinical diagnostics, drug discovery and assessment of therapeutic interventions. In particular, the clinical assessment of sets of multiple protein markers has been associated with an improved understanding of abnormal metabolic state, physiological conditions and diseases.

Enzyme linked immunosorbent assay (ELISA) is a current gold standard protein assaying technique. It usually utilizes paired antibodies along with an enzyme label to generate a colorimetric, quantifiable read out. Various refined, but related assay platforms have been developed. These include methodologies utilizing fluorescence-based signal transduction with enzyme amplification after magnetically separating target proteins (e.g. Simoa® single molecular arrays) and electrochemiluminescence-based immunoassays (e.g. the Meso Scale Discovery (MSD) platform). Although these methods offer high sensitivities, they demand centralized laboratory settings, expensive hardware, technical manipulation and relatively long assay times (6-8 hours), which greatly limit their potential for translation to resource-limited or “point of care” settings.

On the other hand, electrochemical biosensors offer highly sensitive, facile and scalable approaches to protein quantification. A diverse range of microfluidic electrochemical immunoassay protein analysis strategies have been investigated as cost-effective, point-of-care alternatives. Most of these methods can collectively be divided into either labelled, sandwich-type immunoassays or label-free electrochemical immunoassays. Although sandwich-type electrochemical immunoassays promise high sensitivity derived by enzyme catalytic activity or nanoparticle labels, they are inevitably associated with multiple washing and incubation steps, increasing complexity, cost, end user intervention, and time. Furthermore, specifically designed labels—often antibodies conjugated with a detectable tag—are needed for each target molecule, associated with long assay time and high cost due to synthesis, purification and analysis for target affinity.

In contrast to labelled immunoassays, label-free assay methodologies aim directly to transduce the binding event of the protein of interest at an interface decorated with recognition elements into a measurable signal and therefore offer the potential for significant reduction in complexity and costs. However, the effects of non-specific signal suppression arising from the protein matrix associated with “real life” samples is generally more pronounced relative to methodologies involving a labelling step, which often also come with advantages in sensitivity due to the “turn-on” nature of signal generation.

There is therefore a continuing need for new electrochemical assay methods for sensing proteins, which combine properties such as high sensitivity, selectivity, reproducibility, scalability, ease of use, high speed, and low cost into a single platform.

### SUMMARY OF THE INVENTION

The present inventors have developed an assay for electrochemical sensing of proteins that couples non-specific tagging of proteins in a carrier medium with specific (i.e., selective) recruitment and electrochemical detection of a particular target biomarker (i.e., a tagged protein of interest). Such an assay differs from sandwich-type electrochemical immunoassays in that the labelling stage is non-selective and generally applicable in nature. Moreover, it differs from previously described label-free assay methods in relation to the use of the initial “universal” or “shotgun” tagging in the sample, generating a signal increasing with concentration in contrast to many turn-off assays. As further discussed herein, this refined assay technique has been found to yield numerous important practical advantages.

Outside the field of electrochemical biosensing, shotgun protein tagging (where a tag binds non-selectively to disparate proteins in a protein mixture) has been widely used in the field of mass spectrometry. In such methods, proteins are derivatised with either stable isotopes of slightly different masses or isobaric mass tags consisting of multiple isotopic variants yielding different fragment ions in MS/MS, enabling the differentiation of proteins. These tags commonly employ N-hydroxysuccinimide crosslinking groups to target the F-amine group of lysine or N-terminal amine groups released from a protease digestion. Tandem mass tags (TMTs) (which comprise a mass reporter, mass balance group and amine-reactive moieties) and isobaric tags for relative and absolute quantification (iTRAQ) have been among the most abundant chemical labelling methods in MS for intact proteins labelling. A diverse range of quantitative MS applications utilizing such protein tagging techniques have been introduced in the last decade to improve the MS quantitation abilities and to allow mapping of protein structure and interactions. Nonetheless, MS techniques remain expensive, often only semi-quantitative, operationally demanding and far from scalable, in stark contrast to electrochemical biosensing platforms.

Ensemble protein labelling via fluorescent or chemiluminescent probes has also been widely used for protein imaging, mainly inside living cells, and their quantification using fluorescence or luminescence microscopy. A range of aryl-diazonium, azlactone, vinyl sulfone, and NHS-esters/Isothiocyanates bioconjugation strategies have been applied. Fluorescent labelling of protein has evolved from in vivo expression as fusion constructs with fluorescent proteins like green fluorescent protein (GFP) to chemical labelling with fluorescent organic dyes. However, while fluorescent protein tagging has proven a powerful tool for mapping protein expression inside living cells and to follow cellular uptake of tagged proteins, it has not been widely applied for protein quantification. This may be due to the high-end technical requirements and limitations like photo-bleaching and signal-to-noise ratio that render estimation of overall protein concentrations technically demanding. Again, this stands in contrast to the beneficial characteristics of electrochemical biosensing platforms.

The present invention is based, in part, on the recognition that shotgun protein tagging principles, previously applied in quite different and specialized applications such as quantitative mass spectroscopy and cellular fluorescence spectroscopy, could beneficially be translated to electrochemical protein sensing. In particular, in the presently described assays, non-specific protein labelling is effected using a redox active tag that can render the protein molecules susceptible to electrochemical sensing. Surface-specific recruitment of “redox-activated” target protein is then effected and used to sense (e.g., quantify) the target protein by electrochemical means.

The present invention thus provides an electrochemical method of sensing target protein molecules, which method comprises: (A) attaching redox active tagging moieties to protein molecules in a carrier medium that may contain said target protein molecules, wherein the redox active tagging moieties non-specifically bind to protein molecules; (B) contacting the carrier medium with an electrode that comprises receptors that specifically bind to target protein molecules that are bound to the redox active tagging moieties; and (C) electrochemically determining whether target protein molecules are present in the carrier medium.

The present invention also provides a kit for use in the method of the invention, which comprises: (I) redox active tagging moieties that non-specifically bind to protein molecules; and (II) an electrode that comprises receptors that specifically bind to target protein molecules that are bound to the redox active tagging moieties.

Further preferred features and embodiments are described in the accompanying description and the appended claims.

### DESCRIPTION OF THE DRAWINGS

FIG. 1 is a schematic illustration of the materials, systems and methods performed in Example 1.

FIG. 2 shows, with reference to Example 1: in Panel A, Langmuir-Freundlich fit of CRP spiked in 1% human serum in MES buffer (inset is a linear semi-log correlation graph for CRP with correlation coefficient (R²) of 0.993); in panel B, representative DPV peaks as function of increasing CRP concentration on Anti-CRP decorated GCE; in Panel C, Langmuir-Freundlich fit of cTnI spiked in 1% human serum in MES buffer (inset is a linear semi-log correlation graph for cTnI with correlation coefficient (R²) of 0.965); and, in panel D, representative DPV peaks as function of increasing cTnI concentration on anti-cTnI decorated GCE. Error bars represent standard deviations between two independent measurements at two different electrodes.

FIG. 3 shows, with reference to Example 1: in Panel A, an electrochemical specificity study for Anti-CRP modified electrodes in offline single protein analyses; and, in Panel B, an electrochemical specificity study for Anti-cTnI modified electrodes in offline single protein analyses. The response to studied, large excess, interfering species was less than 20% of the target-specific signal indicating a good specificity and low cross-reactivity. Error bars represent standard deviation from two independent measurements. The concentrations used represent estimates for protein content of 10 to 100 times diluted human serum.

FIG. 4 shows, with reference to Example 1, the results of an electrochemical specificity study for Anti-CRP modified electrodes (left-hand bars) as compared with SPR analysis of Anti-CRP modified gold chips (right-hand bars). Left y-axis represents change in peak current and right y-axis represents change in SPR response units (RU) upon exposing the interface to the specified substances. All measurements were in MES buffer. The concentrations used represent estimates for protein content of 10 to 100 times diluted human serum.

FIG. 5 shows, with reference to Example 1: in panel A, Langmuir-Freundlich fits from offline multiplexed protein assay for CRP spiked in 1% human serum in 0.1M MES buffer; and, in panel B, Langmuir-Freundlich fits from offline multiplexed protein assay for cTnI spiked in 1% human serum in 0.1M MES buffer. Insets represent linear semi-log correlation with correlation coefficients (R²) of 0.954 for CRP and 0.939 for cTnI (R²). Error bars represent standard deviations between three independent measurements.

FIG. 6 shows, with reference to Example 1, a schematic representation of the microfluidic chip used for sample handling and electrochemical detection.

FIG. 7 shows, with reference to Example 1, an electrochemical specificity study for anti-CRP modified electrodes (panel A) and anti-cTnI modified electrodes (panel B) in online microfluidic multiplexed protein analyses. The response to studied interfering species was between 5-15% of the target-specific signal indicating a good specificity and low cross-reactivity towards other proteins. Error bars represent standard deviation from two independent measurements. The concentrations used represent estimates for protein content of 10 to 100 times diluted human serum.

FIG. 8 shows, with reference to Example 1, Langmuir-Freundlich fits from online microfluidic multiplex protein assay for CRP (panel A) and cTnI (panel B), spiked both in 1% human serum in 0.1M MES buffer. Insets represent linear semi-log correlation with correlation coefficients (R²) of 0.978 for CRP and 0.993 for cTnI (R²). Error bars represent standard deviations between three independent measurements.

FIG. 9 shows, with reference to Example 1, blind comparison of (A) CRP concentrations and (B) cTnI concentrations estimated using the shotgun protein tagging assay against a reference Immunoassay for 12 patient samples. (Asterisk represent samples where cTnI were lower than the immunoassay LOD i.e <2 pg/mL). Error bars represent standard deviations from two independent measurements on two different electrodes. In each panel left-hand bars for a particular sample number are reported values and right-hand bars for a particular sample number are found values.

FIG. 10 shows, with reference to Example 1, correlation plots between found (A) [CRP] and (B) [cTnI] from 12 patient samples against a standard immunoassay.

### DETAILED DESCRIPTION

Optional and preferred features of the present disclosure are now described. Any of the features described herein may be combined with any of the other features described herein, unless otherwise stated.

### Target Protein Molecules and Carrier Medium

The present disclosure is concerned with the sensing of protein molecules (e.g., determining the presence, or concentration, of such molecules in a carrier medium).

The term protein as used herein encompasses polymeric molecules that comprise a plurality of amino acid residues. Usually a protein molecule comprises at least 5, at least 10, at least 20, or, most commonly, at least 50 amino acid resides. However, unless expressly indicated there is no particular limitation on the lower or upper limit in the number of amino acids comprised by the protein molecules described herein (as long as binding does not affect receptor binding site on the target and moieties for the incorporation of the redox active tag are present within the target protein).

The protein molecules described herein can comprise any natural or non-natural amino acids. For example, a protein molecule may contain only α-amino acid residues, for example corresponding to natural α-amino acids. Alternatively a protein molecule may additionally comprise one or more chemical modifications. For example, the chemical modification may correspond to a post-translation modification, which is a modification that occurs to a protein in vivo following its translation, such as an acylation (for example, an acetylation), an alkylation (for example, a methylation), an amidation, a biotinylation, a formylation, glycosylation, a glycation, a hydroxylation, an iodination, an oxidation, a sulfation or a phosphorylation. It is true that such post-translationally modified proteins still constitute a “protein” within the meaning of the present invention. For example, it is well established in the art that a glycoprotein (a protein that carries one or more oligosaccharide side chains) is a type of protein.

In the methods and kits of the present disclosure, protein molecules are contained in a carrier medium, typically a carrier liquid. The carrier liquid may be any liquid in which protein molecules can be suspended, dissolved, or dispersed. For example, the carrier liquid may comprise water. The carrier liquid may comprise or consist of a biological fluid. A biological fluid may be a fluid that has been obtained from a subject, which may be a human or an animal. In an embodiment, the carrier liquid comprises an undiluted biological fluid. An undiluted biological fluid in the present context is a biological fluid obtained from a subject, e.g. a human or animal, that has not been diluted with another liquid. The biological fluid may be selected from blood, urine, tears, saliva, sweat, and cerebrospinal fluid. Optionally, the carrier medium comprises a biological fluid obtained from a subject, e.g. a human or animal, and a diluent. The diluent may be added to the biological fluid after it has been obtained from the subject. The diluent may include a liquid medium, e.g. a liquid medium selected from water and an alcohol, e.g. an alcohol, e.g. ethanol. The carrier medium may further comprise a buffer. The buffer may comprise phosphate, saline, or other buffer components.

A carrier medium often contains protein molecules that are different from the target protein molecules. By “different” is meant the carrier medium contains (typically a plurality of) molecules of a protein that is chemically different (i.e. has a different chemical structure) from the target protein molecules, and does not merely refer to (physically) different molecules of a single protein. In other words, the carrier medium contains molecules of a protein that is (chemically/structurally) different from the target protein molecules. The carrier medium often comprises a mixture of at least 2, more preferably at least 4, and more preferably still at least 6 different proteins. For instance, the target protein molecules may be present in the mixture together with molecules of at least 1, more preferably at least 3, and more preferably still at least 5 different proteins. Such protein mixtures are, of course, common in samples of clinical interest, such as biological samples. Non-limiting examples of the different proteins that may be present in the mixture together with the target protein molecules include fibrinogen and human serum albumin. Another example of such a different protein is bovine serum albumin.

The target protein molecules are often molecules of clinical interest, for instance as biomarkers that may be useful for monitoring the health of a subject, the presence of a pathological condition, and/or the efficacy of an ongoing or concluded period of therapy for a particular pathological condition. The sensitive detection of biomarkers in physiological samples is of ever growing interest in diagnosis. The present methods can be used in order sensitively and selectively to sense (and determine the concentration) of protein biomarkers, specifically by providing an electrode substrate that is functionalised with receptors that are capable of specifically binding to the biomarker of interest. Examples of proteins of interest include proteins that are a biomarker of one or more of cardiovascular disease (CVD), neurodegeneration, cancer, myocardial infarction, diabetes, pathogenic infection and general trauma.

Strictly non-limiting examples of protein molecules that may be target protein molecules in the methods and kits described herein include dengue NS1 protein; angiotensin I converting enzyme (peptidyl-dipeptidase A); adiponectin; advanced glycosylation end product-specific receptor; alpha-2-HS-glycoprotein; angiogenin, ribonuclease, RNase A family, 5; apolipoprotein A-1; apolipoprotein B (including Ag(x) antigen); apolipoprotein E; BCL2-associated X protein; B-cell CLL/lymphoma 2; complement C3; chemokine (C-C motif) ligand 2; CD 14, soluble; CD 40, soluble; cdk5, pentraxin-related; cathepsin B; dipeptidyl peptidase IV; Epidermal growth factor; endoglin; Fas; fibrinogen; ferritin; growth hormone 1; alanine aminotransferase; hepatocyte growth factor; haptoglobin; heat shock 70 kDa protein 1 B; intercellular adhesion molecule 1; insulin-like growth factor 1 (somatomedin C); insulin-like growth factor 1 receptor; insulin-like growth factor binding protein 1; insulin-like growth factor binding protein 2; insulin-like growth factor-binding protein 3; interleukin 18; interleukin 2 receptor, alpha; interleukin 2 receptor, beta; interleukin 6 (interferon, beta 2); interleukin 6 receptor; interleukin 6 signal transducer (gp130, oncostatin M receptor); interleukin 8; activin A; leptin (obesity homolog, mouse); plasminogen activator, tissue; proopiomelanocortin (adrenocorticotropin/beta-lipotropin/alpha-melanocyte stimulating hormone/beta-melanocyte stimulating hormone/beta-endorphin); proinsulin; resistin; selectin e (endothelial adhesion molecule 1); selectin P (granule membrane protein 140 kDa, antigen CD62); serpin peptidase inhibitor, clade E (nexin, plasminogen activator inhibitor type 1), member 1; serum/glucocorticoid regulated kinase; sex hormone-binding globulin; transforming growth factor, beta 1 (Camurati-Engelmann disease); TIMP metallopeptidase inhibitor 2; tumor necrosis factor receptor superfamily, member 1 B; vascular cell adhesion molecule 1 (VCAM-1); vascular endothelial growth factor; Factor II, Factor V, Factor VIII, Factor IX, Factor XI, Factor XII, F/fibrin degradation products, thrombin-antithrombin III complex, fibrinogen, plasminogen, prothrombin, von Willebrand factor, D-dimer, alpha-synuclein (α-sync), C-reactive protein (CRP); Cardiac Troponin I (cTnI); Troponin T (TnT; TropT); glycated hemoglobin (HbAlc), insulin; TRIG; GPT; HSPA1 B; IGFBP2; LEP; ADIPOQ; CCL2; ENG; HP; IL2RA; SCp; SHBG; TIMP2; Covid-19 S protein; and bacterial biomarker proteins.

### Redox Active Tagging Moieties

The redox active tagging moieties are molecules that have the following features: (a) they are able to non-specifically bind to protein molecules; and (b) they are redox active.

With respect to feature (a), by “non-specifically bind” is meant that the redox active moieties bind not only to a single specific type of protein, but bind to different types of protein; put another way, when contacted with a carrier medium containing at least two different types of protein, the redox active moieties bind to the different types of protein and not merely to (or predominantly to) one particular type of protein or sub-group of the types of protein. “Non-specifically” can be contrasted with “specifically”, particularly in the context of the specific binding exhibited by the receptors in the methods disclosed herein. Non-specific binding of proteins is, of course, well known in the art and its meaning would be readily understood.

In the present disclosure, a typical carrier medium contains protein molecules that are different from the target protein molecules (and the carrier medium may additionally contain target protein molecules). Typically, in such a carrier medium the redox active tagging moieties bind to both: (i) target protein molecules, if present; and (ii) the protein molecules that are different from the target protein molecules. This binding to both (i) and (ii) constitutes one non-limiting meaning for the expression “non-specifically bind(s) to protein molecules”. “Protein molecules” is hence a collective term; it includes both target protein molecules, if present in the sample, and protein molecules that are different from the target protein molecules.

In one exemplary aspect of the invention, the protein molecules that are different from the target protein molecules comprise fibrinogen. In another exemplary aspect of the invention, the protein molecules that are different from the target protein molecules comprise human serum albumin. For instance, the protein molecules that are different from the target protein molecules may comprise both fibrinogen and human serum albumin. In a still further exemplary aspect of the invention, the protein molecules that are different from the target protein molecules comprise bovine serum albumin.

In a typical method of the disclosure, the step (A) of attaching redox active tagging moieties to protein molecules in the carrier medium may comprise contacting the redox active tagging moieties with the carrier medium and, thereby, reacting the redox active tagging moieties with protein molecules therein to form protein molecules that are bound to (and preferably covalently bound to) the redox active tagging moieties.

When the carrier medium contains target protein molecules, step (A) produces target protein molecules that are bound to (and preferably covalently bound to) the redox active tagging moieties. When the carrier medium contains protein molecules that are different from the target protein molecules, step (A) produces protein molecules that: (i) are (preferably covalently) bound to the redox active tagging moieties; and (ii) are different from target protein molecules that are (preferably covalently) bound to the redox active tagging moieties.

The binding of the redox active moieties to protein molecules can be covalent or non-covalent in nature, but preferably is covalent. There is no particular limitation on the precise nature of the binding of the redox active moieties to protein molecules. For instance, the binding may occur through complementary functional groups on the redox active moieties, on the one hand, and the protein molecules, on the other hand. Hence, the redox active moieties preferably comprise a protein-binding portion.

It will be appreciated that particularly suitable strategies for binding the redox active moieties to protein molecules, given the desired non-specific nature of the binding, involve reactions with functional groups that are commonly present, and accessible for reaction, in a wide range of proteins (as usefully contrasted again with the specific binding exhibited by the receptors, which might typically involve, for instance, aptamers or antibodies (or fragments thereof) that recognise and bind, e.g. non-covalently, at least predominantly only to a specific, complementary protein).

For example, non-limiting, suitable binding strategies include those in which the redox active moieties feature, for instance as a protein-binding portion, arenediazonium salts, azlactone groups, vinyl sulfone groups, NHS-ester groups (NHS=N-hydroxysuccinimide) and isothiocyanate groups. Substantially any functional groups commonly found on proteins can be targeted (typical such groups being primary or second amines, such as at the termini of proteins and, especially, on lysine residue side chains, and the thiol groups of cysteine residue).

One exemplary binding strategy, for instance, utilizes an NHS-ester group on the redox active moieties for targeting amines (particularly primary amines such as lysine side chains) on protein molecules. The use of redox active moieties having an NHS-ester group can be particularly convenient owing to the resulting mild coupling conditions and high reaction rate to protein molecules.

In one purely non-limiting aspect of the disclosure, the term “non-specifically” means that the redox active tagging moieties bind to the target protein molecules but also bind to an unrelated control protein (e.g. the “protein molecules that are different from the target protein molecules” described herein, including, but not limited to, fibrinogen, human serum albumin, or bovine serum albumin) such the respective binding affinities are not substantially different. In one further entirely non-limiting example, the unrelated control protein is hen egg white lysozyme.

Preferably, particularly in the atypical situation where the non-specific binding is not covalent in nature, the redox active tagging moieties bind to the target protein molecules with an affinity that is not more than 50, 25, 10, 5 or 2 times greater than (for instance that is substantially no greater than, i.e. that is substantially the same as) the affinity for a control protein (e.g., fibrinogen, human serum albumin, bovine serum albumin, or hen egg white lysozyme). Affinity may be determined by any means known in the art, such as by an affinity ELISA assay, a BIAcore assay, a kinetic method, and/or an equilibrium/solution method. Relative affinity for target protein molecules and control protein can also be measured using well-known competitive binding assays.

Preferably, particularly in the typical situation where the non-specific binding is covalent in nature, under reaction conditions in which the redox active tagging moieties bind (e.g., covalently bind) to the target protein molecules, the redox active tagging moieties also bind (e.g., covalently bind) to a control protein (e.g., fibrinogen, human serum albumin, bovine serum albumin, or hen egg white lysozyme). Such reaction conditions include those typical when performing step (A) of the method of the present invention. For instance, one entirely non-limiting example of such reaction conditions are those in which the redox active tagging moieties are contacted with an optionally buffered (e.g. MES buffer at pH 6.0) aqueous solution comprising both the said target protein molecules and the said control protein at 20° C. With respect to feature (b), by “redox active” means that the redox active moieties are capable of gaining or losing electrons (displaying redox activity) in order to change redox state. The redox active moieties, when bound to target protein molecules that are themselves bound to receptors comprised by the electrode, modulate the electrochemical signal that is obtained in the electrochemical determination step (C) of the disclosed method, this modulation being a consequence of their redox activity. This modulation of electrochemical signal is therefore correlated to the absence, presence and/or concentration of target protein molecules in the carrier medium, in view of the fact that redox active moieties become associated with (indirectly attached, via the receptors and target protein molecules, to) the electrode specifically when there are target protein molecules in the carrier medium.

There is no particular limitation on the structure of the redox active moieties beyond that they exhibit the relevant redox activity. For instance, the redox active moieties may comprise a redox active portion (e.g. as well as comprising a protein-binding portion). For instance, the redox active moieties may comprise, for instance as a redox active portion, a metallic chemical complex comprising a transition metal such as Fe, Ru, Ti, V, Mn, Cr, Co, Ni, Nb or Mo. Further representative examples of suitable redox active species that may constitute a redox-active portion include osmium-based redox active groups, ferrocenes, quinones and porphyrins, including derivatives thereof (e.g., alkyl (e.g., C₁₋₆ alkyl) or acyl derivatives of ferrocene). Derivatives of quinones include p-benzoquinone and hydroquinone. Another currently preferred example of redox active Moieties that may constitute a redox-active portion are those that comprise a redox active portion derived from methylene blue, e.g. containing a structure

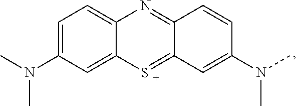

wherein the dashed line indicates a bond (to the remaining part of the redox active moiety).

### Electrode

The electrode functions as the working electrode in an electrochemical system, specifically a system adapted for performing the methods described herein.

Typically the electrode comprises an electrically conductive substrate. This substrate may comprise any electrically conducting material. The substrate may comprise a metal or carbon. The metal may be a metal in elemental form or an alloy of a metal. Optionally, the whole of the substrate comprises a metal or carbon. The substrate may comprise a transition metal. The substrate may comprise a transition metal selected from any of groups 9 to 11 of the Periodic Table. The substrate may comprise a metal selected from, but not limited to, rhenium, iridium, palladium, platinum, copper, indium, rubidium, silver and gold. The substrate may comprise a metal selected from gold, silver and platinum. The substrate may comprise a carbon-containing material, which may be selected from edge plane pyrolytic graphite, basal plane pyrolytic graphite, glassy carbon, boron doped diamond, highly ordered pyrolytic graphite, carbon powder and carbon nanotubes.

In one embodiment, the substrate comprises gold, for example the substrate is a gold substrate. However, it is also possible for the substrate to comprise other materials and so, for instance, in other embodiments, the electrically conductive substrate is not a gold substrate. Non-limiting further examples of suitable electrically conductive substrates include carbon (e.g., graphene), platinum, silver, ruthenium oxide and indium tin oxide (ITO). The electrode can be a screen printed electrode (SPE) substrates (e.g. any of gold, carbon, platinum, silver, ruthenium oxide and ITO SPEs, for instance carbon, platinum, silver, ruthenium oxide and ITO SPEs). The electrode comprising the electrode substrate may, for instance, be one electrode in a multi-electrode array (i.e., a multiplex array).

The electrode surface (i.e., the substrate surface) may be planar, which includes a generally flat surface, e.g. without indentations, protrusions and pores. Such substrate surfaces can be readily prepared by techniques such as polishing with fine particles, e.g. spraying with fine particles, optionally in a sequence of steps where the size of the fine particles is decreased in each polishing step. The fine particles may, for example, comprise a carbon-based material (such as diamond) or a metal oxide (such as alumina), and/or may have particles with diameters of 10 μm or less, optionally 5 μm or less, optionally 3 μm or less, optionally 1 μm or less, optionally 0.5 μm or less, optionally 0.1 μm or less. Following polishing, the substrate surface may be washed, e.g. ultrasonically, optionally in a suitable liquid medium, such as water, e.g. for a period of at least 1 minute, e.g. from about 1 minute to 10 minutes. Optionally, the substrate surface may be washed with an abrasive, e.g. acidic, solution, for example following the polishing and, if used, ultrasonic washing steps. The abrasive solution may comprise an inorganic acid, e.g. H₂SO₄, and/or a peroxide, e.g. H₂O₂, in a suitable liquid medium, e.g. water. Optionally, the substrates can be electrochemically polished, which may follow any steps involving one or more of polishing with fine particles, washing e.g. ultrasonically and/or using an abrasive solution. The electrochemical polishing may involve cycling between an upper and lower potential until a stable reduction peak is reached, e.g. an upper potential of 0.5 V or more, optionally 1 V or more, optionally 1.25 V or more, and a lower potential of 0.5 V or less, optionally 0.25 V or less, optionally 0.1 V or less.

### Receptors

The electrode comprises receptors that specifically bind to target protein molecules that are bound to the redox active tagging moieties.

As disclosed elsewhere herein, the electrode typically comprises an electrically conductive substrate. The electrode thus typically comprises both an electrically conductive substrate and receptors. Clearly, the receptors must be located such that they are able to contact the carrier medium, and hence any target protein molecules bound to redox active tagging moieties (and hence bind thereto) when the method of the disclosure is performed. Additionally, the receptors must be stably associated to the electrically conductive substrate, such that they substantially do not detach from the electrically conductive substrate when the method of the disclosure is performed.

Otherwise, however, there is no particular limitation on the means by which receptors can be associated to an electrically conductive substrate. For instance, receptors may be covalently or non-covalently associated to an electrically conductive substrate, and associated either directly (e.g. via covalent or non-covalent bonding to the electrically conductive substrate itself) or indirectly (e.g. via covalent or non-covalent bonding to an intermediate layer, such as a non-fouling film, conductive polymer film, and/or self-assembled monolayer that is itself disposed on the electrically conductive substrate). As those skilled in the art would be well aware, methods for disposing receptors onto electrodes are well known in the art; any of these well-known and routine techniques can be used without limitation.

As applied herein and unless context dictates otherwise, “receptors” (in the plural) typically refers to a plurality of receptor molecules or moieties of a particular (common) chemical structure. For instance, anti-CRP antibody receptors (as utilized for illustrative purposes in the examples section) comprised by an electrode refers to a plurality of anti-CRP antibodies that are comprised by the electrode. The receptors (and by analogy each receptor molecule/moiety) are capable of specifically binding to target protein molecules that are bound to the redox active tagging moieties.

The term “specifically binds” and similar terms (“specifically binding”, “specifically bind”, etc.) in the context of binding between receptors and target protein molecules that are bound to the redox active tagging moieties means that the receptors bind to target protein molecules that are bound to the redox active tagging moieties with much greater affinity than they bind to an unrelated control protein. The concept of specific binding to proteins is very well known in the art; this term is therefore widely understood and those skilled in the art would have no difficulty, for a particular target protein, in identifying whether a particular receptor satisfies the requirement for having the recited capability of specifically binding thereto. Most commonly the specific binding is not covalent binding, but rather is non-covalent.

However, for avoidance of doubt, the distinction between binding to target protein molecules that are bound to the redox active tagging moieties and binding to an unrelated control protein typically excludes comparison with binding to target protein molecules that are unbound to the redox active tagging moieties (in other words, typically “unrelated control protein” does not include target protein molecules that are unbound to the redox active tagging moieties). As those skilled in the art would of course appreciate, it is possible that a proportion of the protein molecules in the carrier medium may not in practice become attached to the redox active tagging moieties in step (A) of the disclosed methods. However, receptors described herein would often still exhibit specific binding to such (unbound) target protein molecules. Hence, specificity as relating to the binding properties of the receptors means specificity relative to different protein molecules, rather than the same protein molecules, but simply in this unbound state.

As with all binding agents and binding assays, those skilled in the art recognize that the various moieties to which a receptor should not substantially bind in order to be suitable would be exhaustive and impractical to list. Therefore, for receptors disclosed herein, the term “specifically binds” refers to the ability of the receptors to bind (e.g., non-covalently bind) to target protein molecules that are bound (e.g., covalently bound) to the redox active tagging moieties with much greater affinity than they bind (e.g., non-covalently bind) to an unrelated control protein (including such an unrelated control protein that is bound to the redox active tagging moieties). In one entirely non-limiting example, the unrelated control protein is hen egg lysozyme. Another example is milk casein. Another example is fibrinogen. A further example is human serum albumin. A still further example is bovine serum albumin. A further example is any one of these proteins that is bound to the redox active tagging moieties. When carrying out the present disclosure in an embodiment where the carrier medium contains a particular protein that differs from the target protein molecules, then the unrelated control protein may preferably correspond to that particular protein (e.g., if the carrier medium contains fibrinogen and this is not the target protein, then this may be a preferred unrelated control protein; if the carrier medium contains human serum albumin and this is not the target protein, then this may be a preferred unrelated control protein; and if the carrier medium contains bovine serum albumin and this is not the target protein, then this may be a preferred unrelated control protein).

Preferably the receptors bind (e.g., non-covalently) to target protein molecules that are bound to the redox active tagging moieties with an affinity that is at least, 50, 100, 250, 500, 1000, or 10,000 times greater than the affinity for a control protein. The receptors may have a binding affinity for target protein molecules that are bound to the redox active tagging moieties of less than or equal to 1×10⁻¹⁰M, less than or equal to 1×10⁻¹¹M, or less than or equal to 1×10⁻¹² M. Affinity may be determined by any means known in the art, such as by an affinity ELISA assay, a BIAcore assay, a kinetic method, and/or an equilibrium/solution method. Relative affinity for target protein molecules that are bound to the redox active tagging moieties and control protein can also be measured using well-known competitive binding assays.

Examples of suitable receptors include antibodies, antibody fragments, nucleic acids, aptamers, oligosaccharides, peptides and proteins. Preferably, the receptor is selected from aptamers, antibodies, nucleic acids and peptides. More preferably the receptor is an aptamer or antibody, and most preferably an aptamer.

The antibody or the antibody fragment may be selected from one or more of the classes IgA, IgD, IgE, IgG and IgM. In a preferred embodiment, the antibody or antibody fragment is of the IgG type. The antibody or antibody fragment may be derived from a mammal, including, but not limited to, a mammal selected from a human, a mouse, a rat, a rabbit, a goat, a sheep, donkey and a horse. The aptamer may be selected from a peptide aptamer, a DNA aptamer and a RNA aptamer.

Clearly, the choice of receptor for a given electrode is determined by the identity of the target protein. For a particular target protein, a corresponding receptor that is capable of specifically binding thereto should be selected. As one illustrative, and purely illustrative example, if the target species is dengue NS1 protein (significant blood concentrations of which are associated with dengue virus infection), then the receptor should be a substance capable of specifically binding to dengue NS1 protein (or more precisely dengue NS1 protein bound to the redox active tagging moieties), such as a dengue NS1 antibody.

In a typical and preferred embodiment of the present invention, the redox active tagging moieties non-specifically bind covalently to protein molecules, while the receptors specifically bind non-covalently to target protein molecules that are bound to the redox active tagging moieties.

### Electrochemical Method

The method of the disclosure in general comprises at least the steps (A), (B) and (C) as described herein.

As discussed in more detail elsewhere, in step (A) redox active tagging moieties are attached to protein molecules in a carrier medium. In step (B), the carrier medium is contacted with an electrode that comprises receptors. Preferably, step (A) of attaching the redox active tagging moieties to protein molecules in a carrier medium is carried out before the carrier medium is contacted with the electrode in step (B) (i.e. step (A) precedes step (B)). For instance, this may advantageously reduce background signals associated with undesirable binding of the redox tagging moieties to proteins on the electrode surface (e.g., antibodies, any blocking proteins, etc.).

Usually step (C) is carried out after steps (A) and (B) have been performed, as this ensures that the attachment of redox active tagging moieties to protein molecules, and specific binding of receptors to target protein molecules that are bound to the redox active tagging moieties, has occurred prior to the electrochemical determination being performed.

There is no particular limitation on the precise nature of step (C). Rather, electrochemically determining whether target protein molecules are present in the carrier medium can be performed by using any electrochemical method suitable for determining whether a target species is associated with an electrode surface. As those skilled in the art would readily appreciate, the electrochemical determination functions by the modulation of electrochemical signal that results when receptors comprised on the electrode are bound to target protein molecules (that are, themselves, bound to the redox active tagging moieties), as compared with when the receptors are not so-bound. Thus, if the carrier medium does contain target protein molecules then a particular experimental measurement will be obtained. On the other hand the measurement will be different if the carrier medium does not contain target protein molecules. Similarly, changes in the measurement will occur as the concentration of the target protein molecules in the carrier medium changes (typically the signal increasing as the concentration of the target protein molecules in the carrier medium increases). Conveniently, the changes as a function of target protein molecules concentration can be quantified by way of a series of control experiments performed using carrier medium containing known concentrations of target protein molecules, which enables preparation of a calibration curve showing the results as a function of concentration and which can therefore be applied to establish the concentration of target protein molecules in a test carrier medium from the electrochemical measurements made on that system.

Those skilled in the art would furthermore be well aware of the diverse range of electrochemical methods that are well known in the art for detecting such changes in the binding state of receptors comprised on electrodes. Such methods include, without any limitation, voltammetric methods such as differential pulse voltammetric (DPV) methods (including squarewave voltammetric, SWV, methods), in general pulsed voltammetric methods as well as cyclic voltammetry and impedance methods, including measurement of conductance and capacitance properties of the electrode comprising the receptors. For instance, therefore, step (C) can comprise electrochemically determining whether target protein molecules are present in the carrier medium by one or more of voltammetry (e.g. by DPV or SWV), by electrochemical impedance spectroscopy (EIS), by electrochemical capacitance spectroscopy, and the like. One merely exemplary, and non-limiting method involving DPV is exemplified in Example 1.

Such electrochemical methods are also routinely quantitative in nature, i.e. they are capable of distinguishing the extent of binding to receptors on the electrode of target protein molecules, and hence the concentration of the target protein molecules in the carrier medium. Again, this principle is exemplified, in a non-limiting way, in Example 1. Consequently, in a preferred aspect of the disclosure, step (C) comprises determining the concentration of the target protein molecules in the carrier medium.

When carrying out the method of the disclosure it has been found that advantageous limits of detection (“LOD”) can be achieved. For instance, in preferred embodiments the limit of detection of the target protein molecules may be lower than 50 pg/mL, preferably lower than 10 pg/mL and more preferably still lower than 2 pg/mL (e.g. approximately 1 pg/mL or lower).

It has also been found that the methods of the invention can achieve beneficially high sensitivity (i.e., distinguishable changes in measured response as a function of only small changes in target protein concentration).

### Microfluidics and Multiplexing

In one preferred aspect of the methods of the disclosure, the method is carried out in a microfluidic device. For instance, step (A) is performed in a microfluidic mixer and/or steps (B) and (C) are performed in a microfluidic well containing the electrode (and into which fluid from the microfluidic mixer can flow). Further, in the kit of the disclosure, the electrode may be adapted for use in a microfluidic well (e.g. having a size suitable for accommodation in a microfluidic well in a microfluidic electrochemical device).

The method may also be carried out in array format, including in a microfluidic array. Hence, the method may be carried out in an array format in which steps (B) and (C) are carried out at least two times using electrodes comprising receptors that bind specifically to different target protein molecules bound to the redox active tagging moieties, and thereby comprising electrochemically determining whether at least two different target protein molecules are present in the carrier medium (the first iteration of steps (B) and (C) being performed using an electrode that binds specifically to target proteins of a first type, the second iteration of steps (B) and (C) being performed using an electrode that binds specifically to target proteins of a second type, and so on). In one strictly non-limiting example, as exemplified in present Example 1, the at least two different target protein molecules comprise or consist of CRP and cTnI. For instance, in such an embodiment, the first iteration of steps (B) and (C) is performed using an electrode that binds specifically to CRP, and the second iteration of steps (B) and (C) is performed using an electrode that binds specifically to cTnI.

In such a set-up, the method may comprise, in step (A), providing a redox active tagged-carrier medium comprising protein molecules bound to redox active tagging moieties, and which is followed in a step (A2) by dividing the redox active tagged-carrier medium into two or more portions. Each portion is then, separately, subjected to a step (B) and a step (C). Typically, for each portion p₁, p₂, p₃, etc. step (B) comprises contacting that portion with an electrode that comprises receptors r₁, r₂, r₃, etc. that bind specifically to target protein molecules t₁, t₂, t₃, etc. that are bound to the redox active tagging moieties. Hence, step (C) comprises electrochemically determining whether target protein molecules t₁, t₂, t₃, etc. are present in the carrier medium. As such, multiple target protein molecules t₁, t₂, t₃, etc. can be sensed from a single sample of carrier medium, optionally using a single device (such as a single microfluidic device that comprises a microfluidic mixer for preparing the redox active tagged-carrier medium which then forks into two or more microfluidic wells each containing an electrode comprising receptors r₁, r₂, r₃, etc.). This is an especially advantageous feature of the disclosure as it enables a multiplexed target quantification, i.e. the detection of different target proteins as an ensemble boosting the diagnostic value, without the need for different labels, i.e. redox active tag molecules as introduced above, and from one single solution.

### Kit

The kit of the disclosure is suitable for, adapted for, and/or specifically designed for use in the methods of the present disclosure.

The kit comprises redox active tagging moieties that bind non-specifically to protein molecules. These redox active tagging moieties are as defined elsewhere herein. They may be provided in any form, for instance a form suitable for storage. For instance, the redox active tagging moieties may be provided in desiccated and/or freeze-dried form. Alternatively the redox active tagging moieties may be provided (e.g. as a suspension or solution) in a suitable carrier medium, such as a suitable liquid, e.g. in aqueous form.

The kit further comprises an electrode that comprises receptors that bind specifically to target protein molecules that are bound to the redox active tagging moieties. The electrode and the receptors are as defined elsewhere herein.

Optionally, the electrode may be provided in ready-to-use format, in a device such as a microfluidic device, such as for point-of-use applications. In other words, the kit may comprise an electrochemical device that comprises the electrode. The electrochemical device may be a microfluidic device, as described elsewhere herein.

The microfluidic device may comprise a microfluidic mixer for mixing a carrier medium with the redox active tagging moieties. The microfluidic device may further comprise a microfluidic well containing the electrode.

The microfluidic device may be in array format. For instance, the microfluidic device may comprise a plurality of microfluidic wells, each containing an electrode that comprises receptors. The device may comprise at least two microfluidic wells, comprising at least: a first microfluidic well containing an electrode that binds specifically to target protein molecules of a first type; and a second microfluidic well containing an electrode that binds specifically to target protein molecules of a second type. In one strictly non-limiting example, as exemplified in present Example 1, the target protein molecules of a first type are CRP molecules and the target protein molecules of a second type are cTnI molecules.

### Apparatus

In general, the methods of the present disclosure can be conducted on a suitable apparatus. This apparatus comprises an electrochemical spectrometer comprising the electrode as described herein (and which constitutes the working electrode of the spectrometer). The spectrometer typically further comprises a reference electrode and/or a counter electrode.

The apparatus optionally further comprises (a) a receiver configured to receive, from said electrochemical spectrometer, input data comprising measurements made on the electrode; and (b) a processor configured to convert said measurements into output data concerning the presence or absence of the target protein molecules in the carrier medium (e.g., the concentration of the target protein). The receiver and processor can be part of a computer. The functionality of the receiver and processor can be achieved by programming the computer to receive input data from the method and to process these data into the output data as described herein.

### EXAMPLES

### Example 1

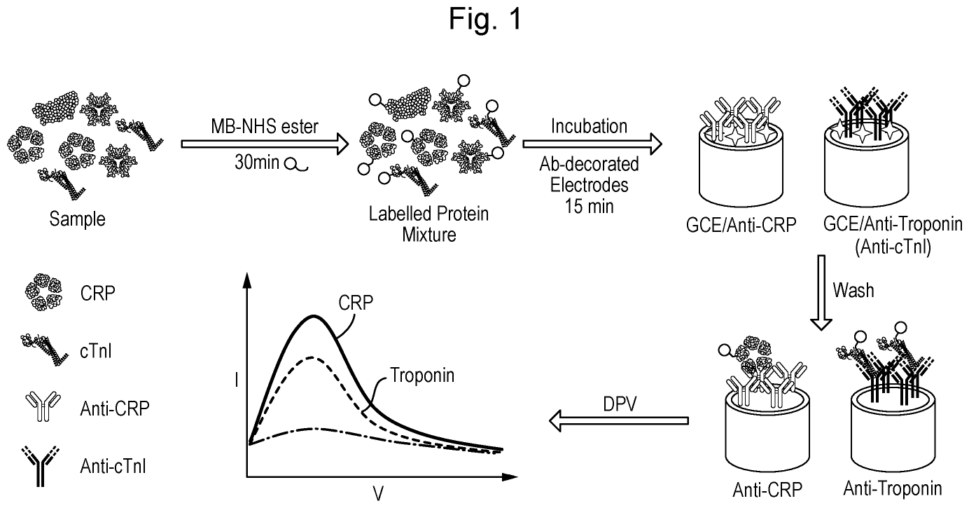

In considering the likely faradaic coupling of any antibody captured redox tagged targets to an underlying electrode, a shotgun succinimide ester tagging of analytical solutions has been utilized prior to immunorecognition mediated voltammetric quantification (FIG. 1). This is a generalizable methodology that supports biomarker assaying at low detection limits (<1 pg/mL) from real biological samples. Further demonstrated is integration of this methodology into a simple microfluidic mixing format that further improves sensitivity and reduces assay times to minutes.

### Results and Discussion

Initially, optimal assay conditions were determined by surveying the faradaic response to targets as a function of buffer pH and methylene blue NHS ester (MB-NHS) concentration, being optimal in MES buffer at pH 6.0, an observation broadly consistent with known succinimide ester coupling efficiencies. Simultaneously, a series of MB-NHS concentrations were tested to achieve maximum signal-to-noise with 10 μg/mL (19 μM in DMSO) being both optimal and generating a dynamic range spanning over 5 orders of magnitude.

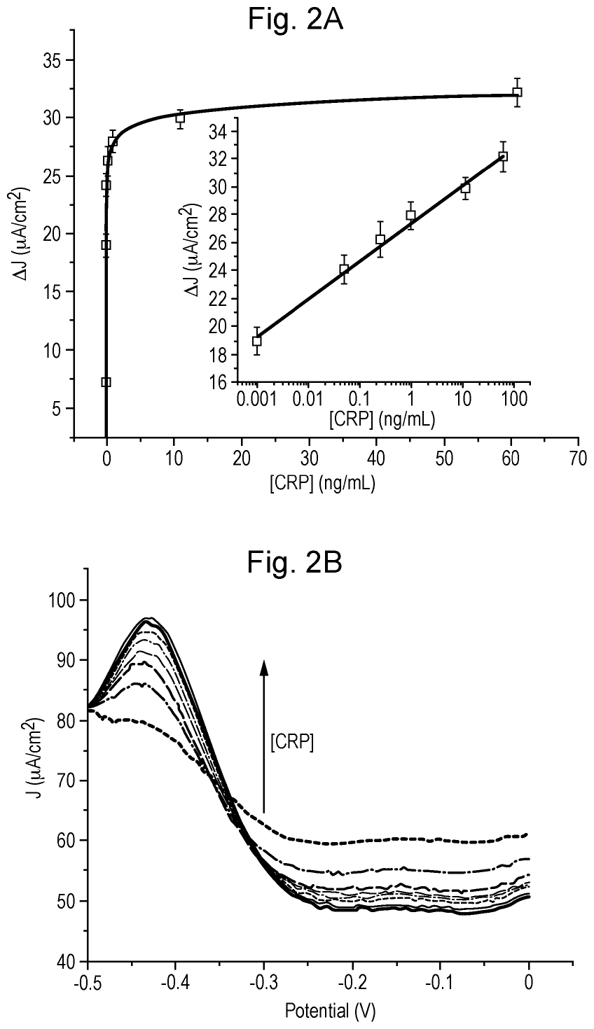

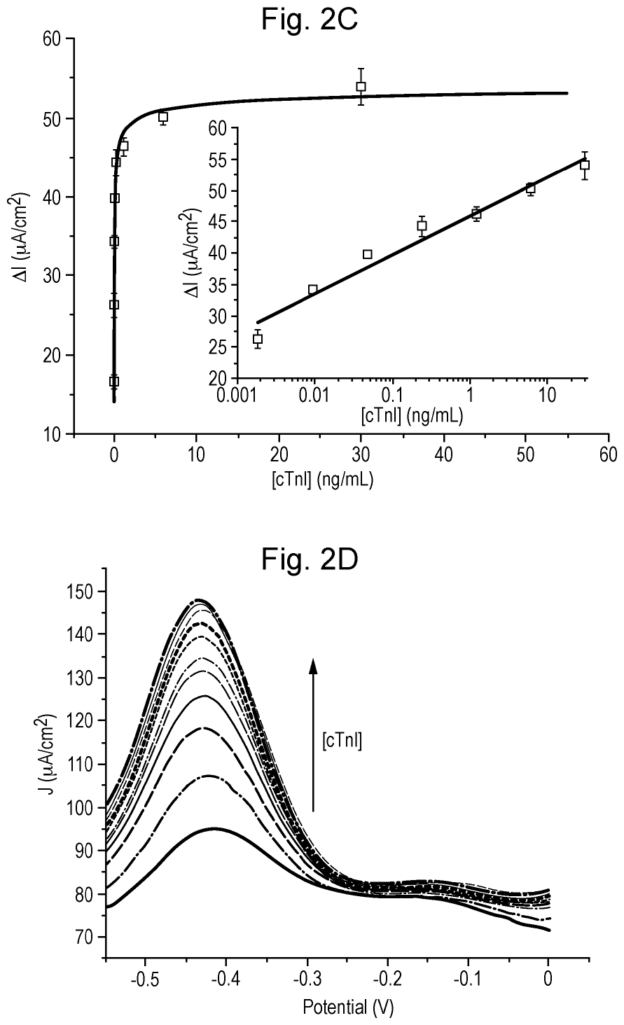

Under these optimized assay conditions, assays were then performed by spiking specific concentrations of either CRP or cTnI into dilute human serum. Samples were tagged by incubating with MB-NHS for 30 minutes after vortexing for 10 sec, prior to reaction quenching through the addition of ethanolamine and hydroxylamine. Labelled samples were then incubated with antibody modified electrodes for 15 minutes prior to DPV sweeps in 0.1M aqueous potassium chloride solution. Good semi-log correlations were observed between specific CRP/cTnI concentrations and DPV peak currents (−0.4 V) with detection limits (LOD) of 1 pg/mL (CRP) and 0.3 pg/mL (cTnI) and dynamic ranges spanning from 1 pg/mL to 100 ng/mL (CRP) and from 0.6 pg/mL to 20 ng/mL for cTnI (FIG. 2)

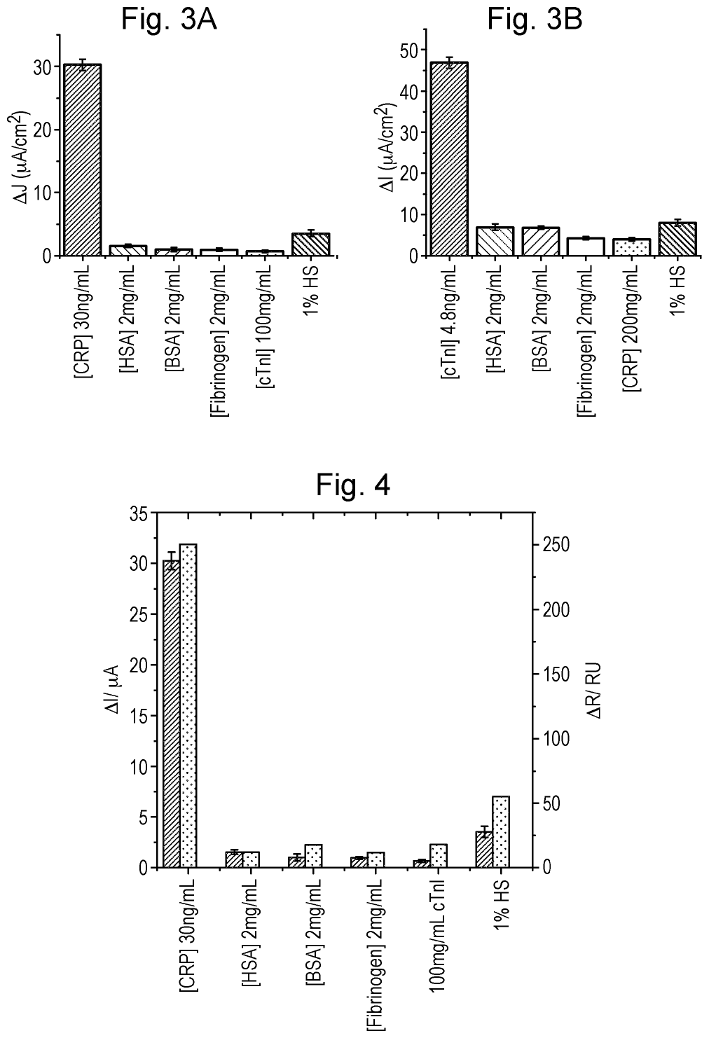

The ability to resolve nonspecific events on the antibody decorated electrodes was assessed by measuring the electrochemical response after challenging the assay against common proteins expressed in human serum like human serum albumin (HSA), fibrinogen, and bovine serum albumin (BSA). In addition, the cross reactivity between the studied proteins was also examined by testing the effect of high concentration of CRP on anti-cTnI modified electrodes and the effect of high concentration of cTnI on anti-CRP modified electrodes. Results, cross referenced to those observed by SPR were indicative of high levels of selectivity (FIG. 3 and FIG. 4).

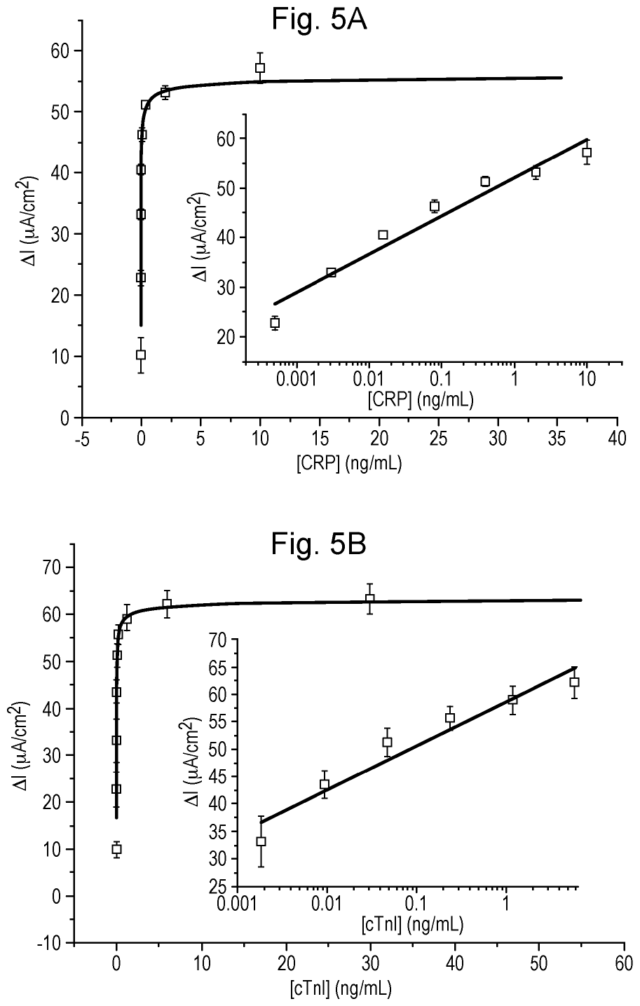

In an assessment of offline sequential multiplexing, MB-labelled mixtures of CRP and cTnI (spiked in 1% human serum) were assessed at two electrodes (one decorated with Anti-CRP and the other decorated with anti-cTnI). The resulting responses (FIG. 5) are very comparable to those observed with single protein spikes, indicating very low levels of interface and antibody cross reactivity.

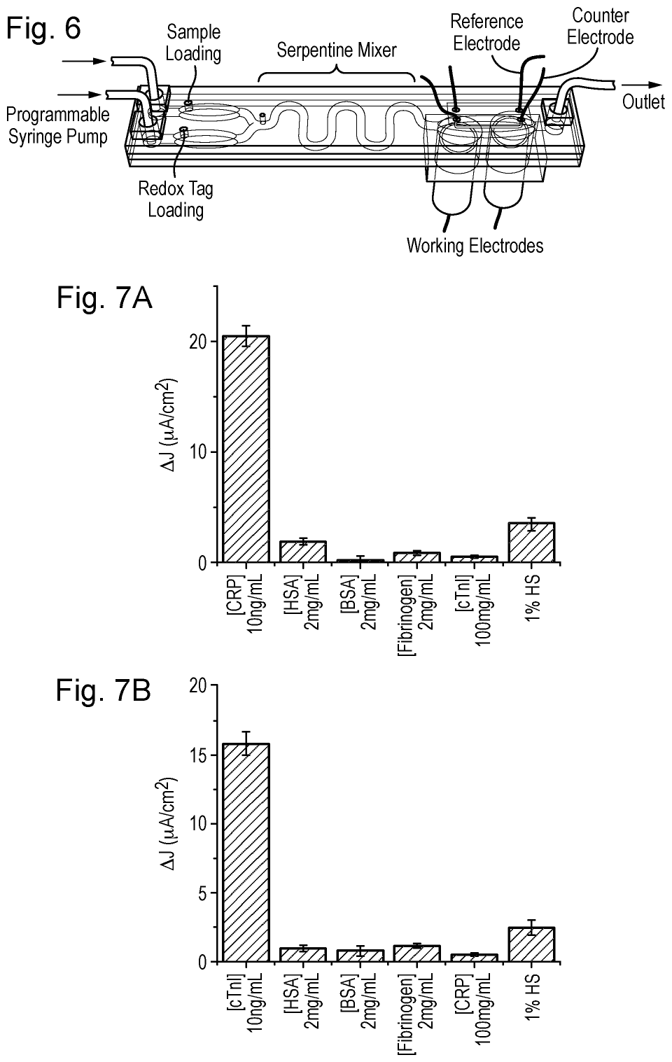

A microfluidic Y-shaped serpentine mixer was then designed and utilized to improve sample/reagent mixing, and analysed in a closed, low volume, chamber directly driven by syringe pump (FIG. 6). The chip houses an electrode compartment that fits two working disc electrodes (outer diameter 6.4 mm, electrode diameter 3.0 mm) with reference Ag/AgCl and counter platinum electrodes connected to a mixing serpentine channel designed to mix sample and reagent pumped from their respective chambers. 40 μL sample and reagent are loaded into their chambers through an injection hole, which is then closed using adhesive tape and connected using flexible tubing to a programmable syringe pump that was programmed to mix the sample and MB-NHS for 5 min, let the mixture incubate in the electrodes chamber for 10 min, followed by 1 min washing.

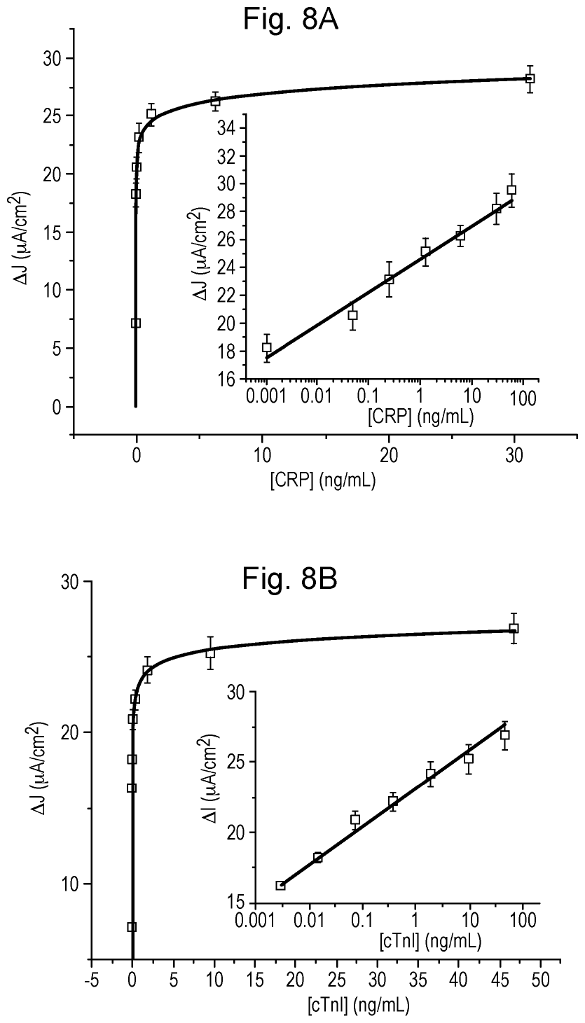

Initial assessments were carried out with CRP alone and then was later extended to detect both CRP and cTnI, simultaneously (FIGS. 7 and 8). The in-line integration of two sensor electrodes supports dual marker quantification (sample-to-answer) within 15 minutes, without the need for quenching or extensive washing procedures; sample and MB reagent loading occurred by pipetting into their respective chambers, prior to mixing by pumping at a pre-optimized flow rate (20 μL/min). The mixture was then incubated at the electrode chamber for 10 min followed by washing by running 0.1M KCl at 100 μL/min for 60 s, and DPV was measured for both working electrodes. Under such conditions, assay specificity was further improved from that observed in the offline/static solution assay as indicated by the notably lower response to interfering species (<10% for both anti-CRP and anti-cTnI modified electrodes). This improved performance within the confines of a flowing microfluidic platform probably arises from a mixture of improved mass transport and a greater suppression of non-specific adsorption as afforded by the enhanced mixing and washing.

The inline microfluidic protein configuration supports very high levels of assay reproducibility and accuracy. Both CRP and cTnI showed a good dose-response with correlation coefficient of 0.978 for CRP and 0.993 for cTnI and dynamic ranges of 1.0 pg/mL-100 ng/mL CRP and 3 pg/mL-62.5 ng/mL cTnI. The standard deviation between three independent measurements were less than 10% indicating a very good reproducibility.

Spiked recovery experiments from dilute (1%) human serum indicate high diagnostic performance (Tables 1 and 2).

TABLE 1
Spike recovery of CRP in 1% Human Serum

| Added conc. (pg/mL) | Recovered conc. (pg/mL) | Recovery % |
|---|---|---|
| 261 | 308 | 118 |
| 661 | 664 | 100 |
| 761 | 762 | 100 |
| 1761 | 1626 | 92 |

TABLE 2
Spike recovery of cTnI in 1% Human Serum

| Added conc. (pg/mL) | Recovered conc. (pg/mL) | Recovery % |
|---|---|---|
| 110 | 118 | 107 |
| 466 | 485 | 104 |
| 1152 | 1095 | 95 |
| 1728 | 1784 | 103 |

### Patient Sample Analysis

The detection of CRP and cTnI simultaneously can greatly reduce mortality and improve treatment outcome for cardiovascular disease (CVD). The analyses above, with detection limits as low as 0.6 pg/mL in serum and dynamic ranges spanning over 6 orders of magnitude compare favourably with most recent electrochemical platforms for sensing of CRP and cTnI, requiring only 25 μL/assay and 15 min or total analytical time (sample dilution to readout; well below recognized guidelines recommending an analysis within one hour of patient admittance, a target not yet reached by currently clinical available methods).

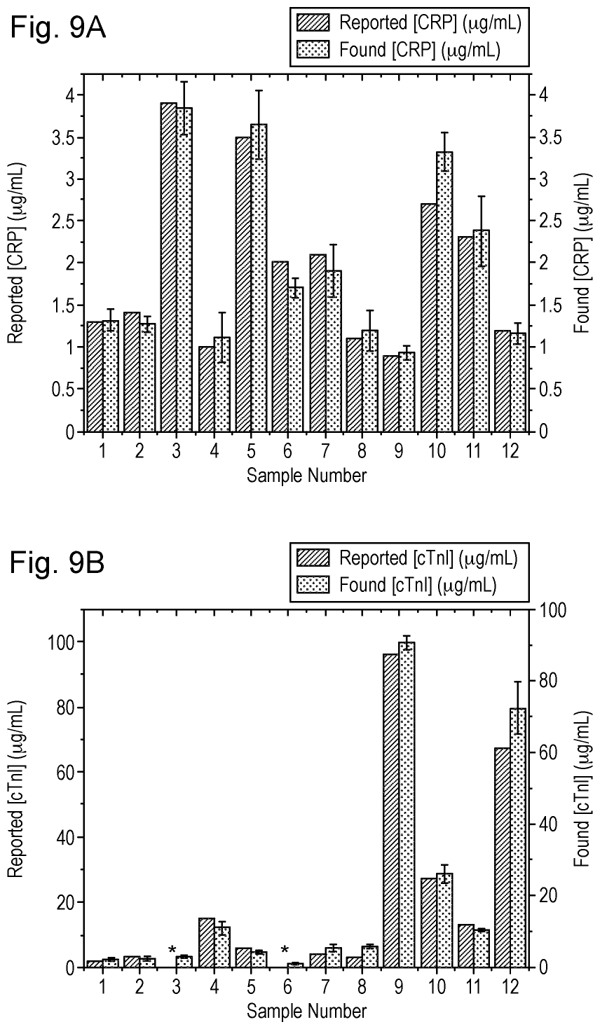

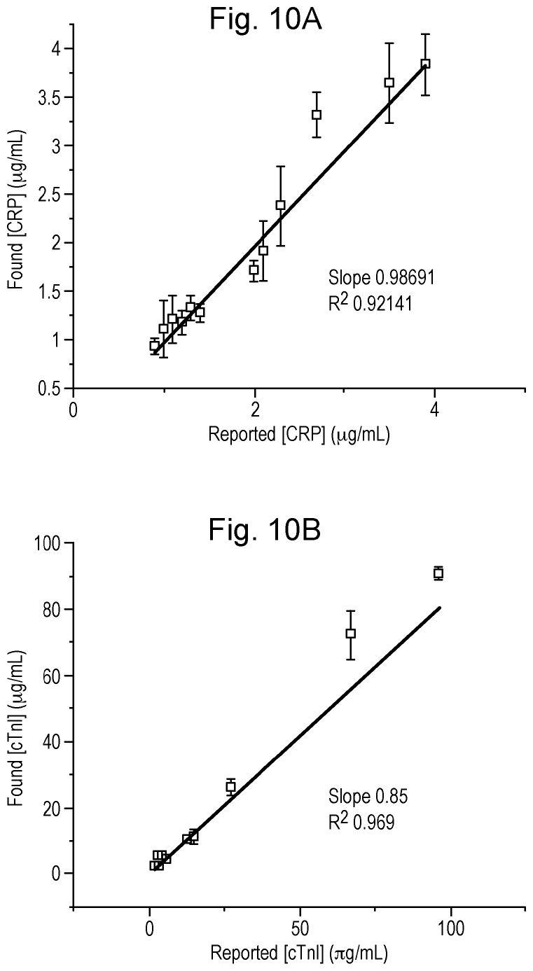

To demonstrate the clinical applicability of the proposed assay, randomized patient samples were analysed and estimated concentrations of CRP and cTnI were compared to immunoassay results from an Abbott Architect system (FIG. 9). Both CRP (FIG. 9A) and cTnI (FIG. 9B) analyses show a good correlation; correlation coefficient and slopes near unity and intercept close to zero (FIG. 10).

### CONCLUSIONS

The results demonstrated the feasibility of applying a solution phase shotgun bulk protein tagging approach for the sensitive electrochemical detection of target proteins. The assay was able to discriminate between tagged target molecule and nonspecific proteins with minimal background interference and high signal/noise ratio especially in human serum. Assay performance was comparable to SPR (as a reference method) results with excellent correlation. The assay showed LOD as low as 0.1 pg/mL in 1% human serum and a dynamic range spanning over 6 orders of magnitude up to 160 ng/mL. Upon integration into a microfluidic cell, volume of the sample required for analysis was less than 25 μL/assay with further potential for reduction; the assay time was ≈15 min. The microfluidic setup carries the potential for full automation and thereby offers a highly scalable and facile design for a biosensor meeting point-of-care demands. The generic approach combines both the advantages in sensitivity and selectivity of labelled assays with the great reduction in complexity offered by labelled approaches promising the possibility of cost-effective application. This work demonstrates the high potential of the proposed novel assay methodology for extremely simple and sensitive detection of protein biomarkers—with diminishing costs and labour requirements. Further, the methodology carries the potential for multiplexed target detection without the need for an additional label by a simple introduction of multiple electrodes or electrode arrays decorated with different antibodies as recognition elements.

### Materials and Methods

If not otherwise stated, all chemicals used were purchased from Sigma-Aldrich. Methylene Blue-NHS ester was purchased from Glen Research and used as provided. Goat anti-human CRP polyclonal antibodies (1707-0189G) and native human CRP (1707-2029) were purchased from BioRad. Anti-cardiac troponin I antibody [M155] (ab10237) and recombinant human cardiac troponin I (cTnI) protein (ab207624) were from Abcam. Water used throughout buffer preparation was ultra-purified with a resistivity of 18.2 MΩ·cm (Milli-Q® Direct/Merck Millipore®). Fibrinogen, bovine serum albumin (BSA), human serum albumin (HAS), fetal bovine serum (FBS) and human serum were purchased from Sigma-Aldrich.

All electrochemical measurements were carried out with a 3 electrode setup using a PalmSens 4 potentiostat powered by PSTrace 5.8. The SPR measurements were performed on Reichert DC7200 using integrated SPRAutolink software. 3D printed microfluidic chips were designed using Fusion 360 software (AUTODESK) and manufactured using an ELEGOO Mars UV photocuring LCD printer with a ELEGOO translucent resin.

Antibody immobilization Gold disk electrodes were polished sequentially with 1.0 μm, 0.3 μm, and 0.05 μm Alumina, sonicated for 10 min in 1:1 H₂O:ethanol and further cleaned with Piranha solution for 5 min. Electrodes were then cycled in 0.5 M sulfuric acid between −0.15 and 1.35 V vs a leak free Ag/AgCl reference electrode (Innovative Instruments Ltd.) at 100 mV/s for 100 cycles. Electrodes were then incubated with a solution of 100 pg/mL of Ab in PBS at pH 7.4 for at least 16 h at 4° C. Glassy carbon electrodes were polished sequentially with 1.0 pam, 0.3 pam, and 0.05 μm Alumina, sonicated for 30 s in 1:1 H₂O:ethanol and cycled in 0.5 M potassium hydroxide between 0.70 and 1.70V vs leak-free Ag/AgCl reference electrode at 100 mV/s for 20 cycles. They were then incubated for 16 h with 100 pg/mL of Ab at 4° C. Electrodes decorated with antibodies were washed with PBS buffer and incubated in 1 M ethanolamine in PBS for 1 h and subsequently with 1% FBS in PBS for 15 min to reduce nonspecific protein binding before measurements.

Solution phase redox tagging 50 p L of freshly prepared methylene blue NHS ester solution (10-100 ug/ml) in DMSO, was immediately mixed with 50 μL of the protein sample aliquoted in either 100 mM MES buffer at pH6.0, 1% FBS in 100 mM MES, or 1% human serum (HS) in 100 mM MES, respectively. The mixture was vortexed and incubated for 30 min without stirring. 20 μL of a mixed solution of 1M Ethanolamine and 1M Hydroxylamine were then added to reaction mixture and incubated for 5 min to quench any unreacted MB-NHS ester.

Electrochemical Measurement Prepared electrodes were incubated with 15 μL of the labelled protein samples for 15 min, washed with PBS, immersed in 0.1M KCl, then DPV curves were recorded between −0.60 and 0.00 V Vs Ag/AgCl reference electrode and platinum (Pt) counter electrode. Electrochemical measurements were performed using PalmSens4 potentiostat and DPV was recorded at 100 mV/s scan rate with 50 mV pulses in 0.01 s.

Specificity study High concentrations of common interfering proteins (2 mg/mL BSA, 2 mg/mL HSA, and 2 mg/mL fibrinogen) were labelled with MB-NHS as described previously and the tagged solutions were then incubated with antibody modified electrodes. DPV signals were recorded and used to compare assay performance against specific response towards target proteins (CRP or cTnI). The cross-reactivity of both target proteins towards their antibodies were also studied by incubating anti-CRP modified electrodes with high concentration of cTnI (100 ng/mL) and incubating anti-cTnI electrodes with high concentration of CRP (200 ng/mL) and comparing the DPV response to the response of each electrode produced by incubation with its perspective target protein.

Design and construction of microfluidic chip The microfluidic chip (FIG. 4 and S3) was designed using the software Fusion360 and subsequently printed using a ELEGOO UV curing 3D printer. A reference electrode was prepared by electrolysis in 0.1M HCl at a plain silver wire (0.25 mm diameter). Subsequently, the so formed reference electrode was coated with Nafion®117 by 8 repeated cycles of incubation in a 4% Nafion solution and air drying. This and a platinum silver wire (0.20 mm diameter) counter electrode were then inserted into the microfluidic cell and fixed via an epoxy resin.

Microfluidic Assay and Measurement An antibody coated electrode was introduced into the microfluidic chip to fit tightly. Inlets of the microfluidic cell were connected to a syringe pump of type Harvard Apparatus Standard Infuse/Withdraw PHD 2200. Before each measurement the microfluidic cell was washed with DI water and dried with nitrogen, then, the two loading chambers (FIG. 4) were filled with 25 μL of 10 g/mL of MB-NHS in DMSO and 25 μL of protein solution, respectively. The loading apertures were then closed using Kapton tape and 0.1M KCl was pumped through both inlets of the cell with a flow rate of 10 μL/min for 5 minutes allowing the two solutions to actively mix inside the flow channel. When the electrode chamber was filled flow was stopped for 10 min to incubate the electrode with tagged protein. Subsequently, the cell was washed with 0.1 M KCl at a flow rate of 100 L/min through each inlet respectively for 2 min. During this the flow cell was installed with a slight positive angle towards the outlet end to prevent the formation of air bubbles. DPV curves were then recorded in 0.1 M KCl between −0.6 and 0.0 V vs Ag/AgCl/Nafion reference electrode with a Pt counter electrode at 100 mV/s scan rate and 50 mV pulses for 0.01 s.

Microfluidic Spike Recovery For the spike recovery experiments in the microfluidic setup, selected known concentrations of both proteins were first analyzed to correct for inter-electrode variations. Electrodes were then exposed to the spiked human serum samples and measured DPV response was used to estimate target protein concentration. Recovery was calculated as the ratio of found concentrations to spiked concentration.

Background correction for microfluidic measurements To account for a changing background current within subsequent DPV measurements at the same electrode in the microfluidic setup, each DPV curve in one set of measurements (subsequent titrations at the same electrode) was background corrected by subtracting the difference between the first points of the blank DPV curve (Ab coated, no incubation) and the curve associated with a specific concentration, respectively. The background correction was chosen due to the lack of a model for a background curve and thereby forces the first points of the curves together to enable the comparison of the peak current across multiple curves. The changing background is suspected to stem from interactions between the reference electrode and DMSO or the fact that not the whole DPV peak can be recorded due to electrode limitations. Relative Response To calculate relative response (RR) from the measured DPV curves upon different titrations, the peak current was divided by the blank current at the peak potential, transformed into a percentage value and subtracting 100% from that according to the formula:

$$RR \% = ( \frac{{I}_{peak}}{{I}_{blank}} - 1 ) \times 100$$

1. An electrochemical method of sensing target protein molecules, which method comprises:
(A) attaching redox active tagging moieties to protein molecules in a carrier medium that may contain said target protein molecules, wherein the redox active tagging moieties non-specifically bind to protein molecules;
(B) contacting the carrier medium with an electrode that comprises receptors that specifically bind to target protein molecules that are bound to the redox active tagging moieties; and
(C) electrochemically determining whether target protein molecules are present in the carrier medium.

2. The method of claim 1, wherein step (C) comprises determining the concentration of the target protein molecules in the carrier medium.

3. The method of claim 1, wherein the carrier medium that may contain said target protein molecules is a biological sample.

4. The method of claim 1, wherein the carrier medium contains protein molecules that are different from the target protein molecules.

5. The method of claim 4, wherein the redox active tagging moieties bind to both: (i) the target protein molecules; and (ii) the protein molecules that are different from the target protein molecules.

6. The method of claim 4, wherein the receptors: (i) specifically bind to target protein molecules that are bound to the redox active tagging moieties; and (ii) do not specifically bind to protein molecules that are different from the target protein molecules and that are bound to the redox active tagging moieties.

7. The method of claim 1, wherein the receptors are selected from the group consisting of aptamers, antibodies, antibody fragments, oligosaccharides, peptides and proteins.

8. The method of claim 1, wherein the receptors are aptamers.

9. The method of claim 1, wherein the target protein molecules are selected from the group consisting of: Covid-19 S protein; dengue NS1 protein; angiotensin I converting enzyme (peptidyl-dipeptidase A); adiponectin; advanced glycosylation end product-specific receptor; alpha-2-HS-glycoprotein; angiogenin, ribonuclease, RNase A family, 5; apolipoprotein A-1; apolipoprotein B (including Ag(x) antigen); apolipoprotein E; BCL2-associated X protein; B-cell CLL/lymphoma 2; complement C3; chemokine (C-C motif) ligand 2; CD 14, soluble; CD 40, soluble; cdk5, pentraxin-related; cathepsin B; dipeptidyl peptidase IV; Epidermal growth factor; endoglin; Fas; fibrinogen; ferritin; growth hormone 1; alanine aminotransferase; hepatocyte growth factor; haptoglobin; heat shock 70 kDa protein 1 B; intercellular adhesion molecule 1; insulin-like growth factor 1 (somatomedin C); insulin-like growth factor 1 receptor; insulin-like growth factor binding protein 1; insulin-like growth factor binding protein 2; insulin-like growth factor-binding protein 3; interleukin 18; interleukin 2 receptor, alpha; interleukin 2 receptor, beta; interleukin 6 (interferon, beta 2); interleukin 6 receptor; interleukin 6 signal transducer (gp130, oncostatin M receptor); interleukin 8; activin A; leptin (obesity homolog, mouse); plasminogen activator, tissue; proopiomelanocortin (adrenocorticotropin/beta-lipotropin/alpha-melanocyte stimulating hormone/beta-melanocyte stimulating hormone/beta-endorphin); proinsulin; resistin; selectin e (endothelial adhesion molecule 1); selectin P (granule membrane protein 140 kDa, antigen CD62); serpin peptidase inhibitor, clade E (nexin, plasminogen activator inhibitor type 1), member 1; serum/glucocorticoid regulated kinase; sex hormone-binding globulin; transforming growth factor, beta 1 (Camurati-Engelmann disease); TIMP metallopeptidase inhibitor 2; tumor necrosis factor receptor superfamily, member 1 B; vascular cell adhesion molecule 1 (VCAM-1); vascular endothelial growth factor; Factor II, Factor V, Factor VIII, Factor IX, Factor XI, Factor XII, F/fibrin degradation products, thrombin-antithrombin III complex, fibrinogen, plasminogen, prothrombin, von Willebrand factor, D-dimer, alpha-synuclein (α-sync), C-reactive protein (CRP); Cardiac Troponin I (cTnI); Troponin T (TnT; TropT); glycated hemoglobin (HbAlc), insulin; TRIG; GPT; HSPA1 B; IGFBP2; LEP; ADIPOQ; CCL2; ENG; HP; IL2RA; SCp; SHBG; bacterial biomarker protein; and TIMP2.

10. The method of claim 1, wherein the redox active tagging moieties comprise a redox active portion and a protein-binding portion.

11. The method of claim 1, wherein the redox active tagging moieties comprise a redox active portion that is selected from: a group derived from methylene blue; a quinone; and a metallic chemical complex comprising a transition metal, wherein optionally the transition metal is Fe, Ru, Ti, V, Mn, Cr, Co, Ni, Nb, Mo or Os.

12. The method of claim 1, wherein the redox active tagging moieties comprise a protein-binding portion selected from an arenediazonium group, an azlactone group, a vinyl sulfone group, an NHS-ester group and/or an isothiocyanate group.

13. The method of claim 1, wherein said electrochemically determining in step (C) comprises determining whether target protein molecules are present in the carrier medium by differential pulse voltammetry.

14. The method of claim 1, which is carried out in a microfluidic device.

15. The method of claim 1, which is carried out in an array format in which steps (B) and (C) are carried out at least two times using different electrodes that specifically bind, respectively, to different target protein molecules bound to the redox active tagging moieties, and thereby comprising electrochemically determining whether at least two different target protein molecules are present in the carrier medium.

16. A kit for use in the method of claim 1, which comprises:
(I) redox active tagging moieties that non-specifically bind to protein molecules; and
(II) an electrode that comprises receptors that specifically bind to target protein molecules that are bound to the redox active tagging moieties.

17. The kit according to claim 16, which comprises a microfluidic device that comprises the said electrode as a working electrode.

18. The kit according to claim 17, wherein the microfluidic device further comprises a counter electrode and/or a reference electrode.

19. The kit according to claim 17, wherein the microfluidic device further comprises a microfluidic mixer.

20. The kit according to claim 16, which comprises two or more different electrodes, wherein each of said different electrodes comprises receptors that specifically bind, respectively, to different target protein molecules bound to the redox active tagging moieties.
